(19) United States
(12) Patent Application Publication
(10) Pub. No.: US 2012/0308985 A1
(43) Pub. Date: Dec. 6, 2012

(54) Immature Reticulocyte Fraction Reference Control and Related Methods

(75) Inventors: Wayne L. Ryan, Omaha, NE (US); John W. Scholl, Omaha, NE (US)

(73) Assignee: STRECK, INC., LaVista, NE (US)

(21) Appl. No.: 13/452,094

(22) Filed: Apr. 20, 2012

Related U.S. Application Data
(60) Provisional application No. 61/477,893, filed on Apr. 21, 2011.

(51) Int. Cl. C12N 5/078

(52) U.S. Cl. 435/2; 435/325; 435/372

(57) ABSTRACT

A composition (and associated methods) including a plurality of treated red blood cells for simulating reticulocytes, and particularly an immature reticulocyte fraction, of whole blood when processed as a sample in an automated analyzer capable of detecting reticulocytes. A method for making the composition or other simulated reticulocyte may include steps of contacting a suspension of a plurality of red blood cells each having a membrane in an initial state that surrounds an interior volume of a cell with an effective amount of a hypertonic permeabilizing solution including dimethyl sulfoxide and a hypotonic loading agent delivery solution including a loading agent, for a sufficient time to form a plurality of pores in the membrane, for permitting the loading agent to enter into the interior volume of the cells.

### CLAIM OF PRIORITY

The present application claims the benefit of the filing date of U.S. Provisional Patent Application No. 61/477,893, filed on Apr. 21, 2011, the contents of which is incorporated herein by reference in its entirety.

### FIELD OF THE INVENTION

This invention relates generally to hematology controls, and more specifically to synthetic stable controls for simulating an immature reticulocyte fraction of blood.

### BACKGROUND OF THE INVENTION

In the field of hematology, detection and enumeration of cells has long been a means for identifying the presence or condition of certain disease states. Analysis has been undertaken manually, such as by microscopy. Automated analysis, such as through the use of hematology analyzers, also has been employed. In more recent years, particularly as automated analytical systems have improved, laboratories have turned their attention to the analysis of reticulocytes, including a population of reticulocytes within the general population of reticulocytes known as the immature reticulocyte fraction (“IRF”). As used herein, the clause “immature reticulocyte fraction” (“IRF”) refers to the ratio of young or immature reticulocytes to the total number of reticulocytes. Immature reticulocytes are larger and are classified as haying the greatest staining or light scatter properties, therefore, the highest level of RNA.

Only the detection analysis of the cytometric methods can quantify the differences in staining. This degree of differentiation in staining cannot be determined by manual methods of evaluation. As a result, a reference method for determining abnormal IRF values has not been established and instrument manufacturers have independently written software algorithms to determine the IRF value specific to their reagent/methodology.

Unfortunately, this has resulted in discrepancies in IRF recovery between instrument manufacturers and even within analyzer systems of the same manufacturer. In addition, the IRF value recovery is dependent upon proper laser alignment to insure proper positioning of the reticulocyte population that determines the correct gating. Unfortunately, as will be discussed, IRF data from automated analyzers have yet to be relied upon extensively by medical practitioners. The following tables demonstrate differences in reticulocyte % and IRF recoveries and run-to-run variations of patient blood between the various instrument manufacturers. The recovery differences are a result of the software differences and lack of reference methods.

Reticulocyte Percent

| Instrument | Ave | SD | CV % |
|---|---|---|---|
| Abbott Sapphire | 6.18 | 0.152 | 2.46% |
| Sysmex XE5000 | 4.50 | 0.157 | 3.48% |
| Siemens Advia | 4.13 | 0.177 | 4.28% |
| 120 | | | |
| Beckman Coulter | 4.65 | 0.279 | 6.01% |
| LH750 | | | |

Immature Retic Fraction

| Instrument | Ave | SD | CV % |
|---|---|---|---|
| Abbott Sapphire | 0.483 | 0.006 | 1.25% |
| Sysmex XE5000 | 0.274 | 0.016 | 6.01% |
| Siemens Advia | 0.291 | 0.009 | 3.15% |
| 120 | | | |
| Beckman Coulter | 0.460 | 0.021 | 4.50% |
| LH750 | | | |

The extent of maturation of a reticulocyte has been identified as important in monitoring certain patient conditions. For example, it has been analyzed for monitoring anemia. It has been analyzed for monitoring the efficacy of erythropoiesis that any treatment has produced. It has been employed to confirm bone marrow regeneration in response to transplant or chemotherapy treatments. It has even been used to assist determination of timing for stem cell harvesting. See generally, Dunlop et al, “The Immature Reticulocyte Fraction: A Negative Predictor of the Harvesting of CD34 Cells for Autologous Peripheral Blood Stem Cell Transplantation”, Clin. Lab. Haem. 2006, 28, 245-247; Naronha et al., “Immature Reticulocytes as an Early Predictor of Engraftment in Autologous and Allogenic Bone Marrow Transplantation,” Clin. Lab. Haem. 2003, 25, 47-54; and Barnes et al, “News from Hematology,” April 2002 Pathology and Laboratory Medicine Newsletter by Childrens Mercy Hospitals & Clinics.

It is known that newly produced reticulocytes generally contain a certain content of ribonucleic acid (RNA) within its membrane covering. With passage of time, the RNA content will reduce, and one or more detectable aspects of the cell will change also. Typically, for a given reticulocyte population, the IRF can be distinguished from the more mature reticulocytes by the differing RNA amounts. By way of example, it is believed that due to an increased presence of RNA, they are more susceptible to binding to certain dyes, certain of which may be fluorescent dyes. Certain automated analyzers will detect such dyes, (e.g., by fluorescence detection, by light scatter, by light absorbance, or otherwise) and display it as a relatively discrete fraction within the reticulocyte fraction. For example, dyes employed may be new methylene blue, Oxazine 750 perchlorate dye, polymethine, or other dyes. See generally, Piva et al, Review, Automated Reticulocyte Counting: State of the Art and Clinical Applications in the Evaluation of Erythropoiesis, Clin. Chem. Lab. Med. 2010; 48(10): 1369-1380.

Without limitation, examples of commercially available automated hematology analyzers include the Sysmex XE5000 instrument and the Abbott Sapphire instrument, Siemens Advia 2120 instrument, and BeckmanCoulter LH-series instruments. See also, Published US Application Nos. 20100240055 and 20100075369, and BeckmanCoulter Technical Information Bulletin No. 9231 “The LH Reticulocyte Count and Associated Parameters”; and Kessler et al, “Immature Reticulocyte Fraction and Reticulocyte Maturity Index” (see, http://www.beckmancoulter.com/literature/ClinDiag/reticliterature.pdf).

As mentioned, IRF data has not been relied upon extensively, to date. One possible explanation is that there is an inconsistency, and lack of standardization among the various automated systems. For example, it is believed that reliance upon IRF data from the use of automated analyzers is potentially impaired by a perceived lack of consistency among instruments. Widely, it has been reported that, as among the various instruments there are different detection strategies employed across the range of analyzers, differences in reagents, differences in algorithms to analyze data, and differences in reference ranges. Accordingly, standardization has been difficult, as discussed in C. Briggs, “Quality Assessment for New Blood Cell Counts”. Int. Jnl. Lab. Hem. 2009, 31, 277-297; and Buttarello et al, “Automated Blood Cell Counts”, Am. J. Clin. Pathol., 2008; 130:104-116. See also, Buttarello and Plebani, “Automated Blood Cell Counts”, Am. J. Clin. Pathol. 2008; 130:104-116. What may show up in one instrument as an IRF, may show up as a mature reticulocyte fraction in another. As a result, data reported about IRF generally has not been relied upon by medical practitioners in the diagnosis and/or treatment of afflicted patients. In view of the ability of many modern automated analyzers to detect and report IRF, it is unfortunate that such feature to date has not seen more consistent usage.

To also illustrate analytical strategies, U.S. Patent Application No. 20100075369 further suggests a methodology by which IRF is determined by the number of reticulocyte events in each of ten defined regions. The IRF is then reported on the basis of the selection of affected regions according to an empirically determined polynomial curve and determining the ratio of reticulocyte events in those regions relative to the total reticulocyte events.

One prior effort to provide reticulocyte analogs (i.e., simulated reticulocytes) is illustrated in U.S. Pat. No. 5,432,089 (Ryan). That patent describes a methodology by which erythrocytes are loaded with a nucleic acid (e.g., ribonucleic acid (“RNA”)) by a reverse osmosis process. In general, the process includes an osmotic lysis process carried out by first washing packed RBCs in an isotonic solution. Next, RNA is added to the RBC solution along with a hemolysate, the solution is mixed to form a suspension, and then a dialysis chamber is prepared with a hypotonic solution. The RBC suspension, once placed in a dialysis bag, is put into the hypotonic solution. When the osmolality of the dialysis bag is about 180 mOsm/Kg, the hypotonic solution is discarded and the RBC suspension is allowed to equilibrate at room temperature. Next, a hypertonic solution is placed in the dialysis chamber, and the RBCs in the dialysis bag undergo resealing of their cell membranes. The resealing step is stopped after isotonicity is restored. The process has certain potential limitations, such as increased cell fragility and smaller cell size. That is, the processing of cells to attain simulated IRF is not a simple and predictable extension of the teachings of this patent in view of the need to introduce a comparatively large amount of RNA to simulate RNA of ah immature reticulocyte, and the need for the cell into which the loading agent is introduced to withstand the necessarily harsh and rapid treatment conditions to achieve the loading and re-sealing of cells.

Another example of the manufacture of reticulocytes, involving maturation arrested porcine cells, is illustrated in U.S. Pat. Nos. 5,945,340; 5,858,789; and 5,736,402 (Francis and Johnson). See also, U.S. Pat. No. 6,444,471 (Johnson).

Still another approach has been illustrated in U.S. Pat. No. 7,195,919, in which an erythrocyte is coated on an external surface with a bio-polymer (e.g., RNA).

The teachings in the above publications that pertain to simulating reticulocytes for a reticulocyte control do not necessarily lend themselves well for making bells for simulating an IRF, in which precise control over the number of cells having the known characteristic is necessary, as well as precise control over the amount of an additive for simulating RNA encapsulated in a reticulocyte of an IRF.

U.S. Pat. No. 7,618,821 addresses the preparation of analogs in which solutions employing dimethyl sulfoxide are used. See also, US Application No. 20100285560.

Other publications in the art that may relate to the present teachings include W. Check, “Perks Plus: The New Hematology Analyzers”, Cap Today (June 2002); Briggs et al, “Comparison of the Automated Reticulocyte Counts and Immature Reticulocyte Fraction Measurements Obtained with the ABX Pentra 120 Retic Blood Analyzer and the Sysmex XE-2100 Automated Hematology Analyzer”, Laboratory Hematology 7:75-80 (2001); and Sandhaus, “How Useful Are CBC and Reticulocyte Reports to Clinicians?”, Am. J. Clin. Pathol. 2002; 118:787-793.

Accordingly, the art needs improved controls and methods for helping to attain improved reliability of IRF detection.

### SUMMARY OF THE INVENTION

By way of summary, the present teachings meet one or more of the above needs by providing a control system that simulates one or more detectable characteristic of a reticulocyte population, and particularly an IRF. The system contemplates a relatively long-term (e.g., at least 12 hours, 24 hours, 48 hours, 72 hours, 1 week, 30 days, 45 days, 60 days, 90 days or longer) storage stable control composition that employs stabilized blood cells that include an outer membrane layer that substantially encapsulates an amount of RNA selected for simulating one or more detectable characteristics (e.g., the size, stainability and/or morphology) of reticulocytes in an IRF.

In a first aspect, the teachings herein pertain to a composition, comprising a plurality of treated red blood cells for simulating varying IRF ranges of whole blood when processed as a sample in an automated analyzer capable of detecting reticulocytes. The treated red blood cells may be of human red blood cell origin. The treated red blood cells may include a synthetically encapsulated loading agent. For example, the treated red blood cells include a synthetically encapsulated polyanionic loading agent capable of binding the instrument reticulocyte stain such as, but not limited to, RNA. The composition may be substantially free, of free hemoglobin. It may be storage stable for a period of at least about 12 hours, 24 hours, 48 hours, 72 hours, 1 week, 30 days, 45 days, 60 days, 90 days or longer.

The composition may include one or more diluents (e.g., a final diluents within which simulated blood components are suspended), which may itself include at least one stabilizing agent present in a sufficient amount for stabilization. Stabilization may be of one or more components, such as the simulated red blood cell component of the composition, so that any such components provide consistent and reproducible readings from an automated analyzer during the period of storage stability.

The stabilizing agent may be selected from a suitable carboxylic acid. For example, it may be selected from a salicylic acid (e.g., sulfasalazine such as in an amount of about 1 to about 25 mg %, e.g., about 10 mg %). It may be selected from one or more of [[[(2-dihydro-5-methyl-3(2H)-oxazolyl)-1-methylethoxy]methoxy]methoxy]methanol (e.g., NuoSept 145), sodium hydroxymethylglycinate (e.g., Suttocide or Nuosept 44), an agent including one or more derivatives of or ingredients having 4,4-Dimethyl-1,3-oxazolidine (e.g., Oxaban A, Nuosept 101 or Nuosept 166) or any combination thereof. Examples of particular preferred agents include [[[(2-dihydro-5-methyl-3(2H)-oxazolyl)-1-methylethoxy]methoxy]methoxy]methanol (e.g., NuoSept 145), in an amount of about 0.2 to about 0.8 v/v % of a suitable post-encapsulation solution (see, e.g., Table 6). Another is sulfasalazine, such as in an amount of about 1 to about 25 mg %, e.g., about 10 mg % of the suitable post-encapsulation solution (see, e.g., Table 6).

Any combination of the above stabilizing agents may be employed. Though it is described that a formaldehyde-donor agent may be employed, the compositions herein may be free of any free formaldehyde, and/pr they may be processed in the absence of any formaldehyde as the starting material for the stabilizing agent.

The composition may exhibit an immature reticulocyte fraction in a known predetermined relative range of amounts of simulated immature reticulocytes. For example, it may have a known range of amounts of simulated immature reticulocytes in a relatively low amount, a relatively high amount, and/or optionally a relatively intermediate, amount of an overall simulated reticulocyte population. As will be appreciated, these values are illustrative, and higher and/or lower values are also possible. The composition may be part of a kit that includes two, three or more different compositions, each having known relative ranges of amounts of simulated immature reticulocytes. The composition may be part of a kit that includes two, three or more different compositions, each with a known range of amounts of simulated mature reticulocytes in combination with other known relative ranges of amounts of simulated immature reticulocytes.

Another aspect of the teachings herein contemplate a method for making a simulated reticulocyte, comprising: contacting a suspension of a plurality of red blood cells each having a membrane in an initial state that surrounds an interior volume of a cell with an effective amount of a hypertonic permeabilizing solution including dimethyl sulfoxide and a hypotonic loading agent delivery solution including a loading agent, for a sufficient time to form a plurality of pores in the membrane, for permitting the loading agent to enter into the interior volume of the cells, and, after entry of a desired amount of the loading agent into the interior volume of the cell, for sealing the pores, for substantially restoring the membrane to the initial state while substantially encapsulating the loading agent within the resulting cell. The processing may also be performed so that a substantial amount of hemoglobin from the original blood cell starting material is maintained, within the resulting cell. For example, it is believed possible that the amount of remaining hemoglobin may be at least the amount, and preferably at least 10% by volume greater, 20% by volume greater or higher than the amount of hemoglobin that is in cells processed in accordance with the teachings of U.S. Pat. No. 5,432,089 (Ryan).

The method may include a step of separating a plurality of human red blood cells from a supply of human red blood cells. For example, the method may includes a step of separating a plurality of human red blood cells from a supply of human red blood cells by contacting the supply of human red blood cells with a stress solution (e.g., a hypotonic stress solution) in an amount and for a time sufficient for selectively destroying weakened or aged red blood cells within the supply. The method may include a step of separating cells by filtering them through a filter (e.g., a leukocyte removal filter). The method may include a step of contacting the plurality of red blood cells with a substantially pH neutral and substantially isotonic preservative diluent for a period of about 5 days to about 30 days. For example, the method includes contacting the plurality of red blood cells with a substantially pH neutral and substantially isotonic preservative diluent for a period of about 5 days to about 30 days, the diluent including EDTA, and being held in a diluted red blood cell concentration of about 1×10⁶ to about 3×10⁶/μl. The method may include a step of packing the plurality of red blood cells to a hematocrit value of about 65 to about 85% in a unit volume of an isotonic solution. The permeabilizing solution may include about 0.05 to about 2 (e.g., about 0.1) parts by volume of solution containing dimethyl sulfoxide. The loading agent delivery solution may be a hypotonic solution and includes about 3 to about 5 (e.g., about 4) parts by volume of a solution including a polyanionic loading agent capable of binding the instrument reticulocyte stain such as, but not limited to, RNA. The loading agent delivery solution may include one or a combination of neomycin sulfate or tris. The step of contacting may include first contacting with the permeabilizing solution and then contacting with the loading agent delivery solution. The method may include a step of removing free hemoglobin and resulting in intact cells in a final solution.

In other aspects, the teachings herein pertain to a reference control that is employed for comparing with a patient sample of blood, to ascertain if an analyzed patient sample results in information about the sample (e.g., intensity amount or both) pertaining to detected reticulocytes that would correspond with information about simulated reticulocytes of the IRF reference control of the present teachings. For example, the present teachings contemplate a method for identifying a condition indicated by an abnormal presence of immature reticulocyte fraction, comprising the steps of: passing a sample of patient blood through an analyzer that detects reticulocytes; compiling patient blood sample information about the presence of reticulocytes including the IRF fraction in the patient blood sample using the analyzer; passing at least one sample of at least one control composition through the same analyzer; compiling control composition sample information about the presence of immature reticulocyte fraction in the control composition; comparing the patient blood sample information with the control composition sample information to identify the extent of overlap of IRF; and (optionally) reporting the results of the comparing step.

In other aspects, the teachings herein pertain to a reference control for assuring consistent and reproducible values for simulating an immature reticulocyte fraction of whole blood. The teachings also pertain to use of such a control, such as in a method for determining the accuracy and reproducibility of the operation of an analytical instrument capable of measuring immature reticulocyte fraction. For example, such a method may include steps of: passing a known quantity of a control through an automated analyzer adapted to be capable of measuring immature reticulocyte fraction; determining the immature reticulocyte fraction level in said control using the instrument; and comparing the immature reticulocyte fraction level obtained with its known reference quantity to ascertain if the instrument is properly functioning. Such analyzers may be configured for detecting reticulocytes bound with a fluorescent dye, or for detecting reticulocytes stained with one or more of new methylene blue, Oxazine 750 perchlorate dye, polymethine, or some other dye.

### DESCRIPTION OF THE DRAWINGS

FIGS. 1a-1c are illustrative scattergrams to show likely expected results from different respective analyzers in which the control compositions have a relatively high IRF.

FIGS. 2a-2c are illustrative scattergrams to show likely expected results from different respective analyzers in which the control compositions have a relatively low IRF, and are prepared from a tris compound containing loading agent delivery solution.

FIGS. 3a-3c are illustrative scattergrams to show likely expected results from different respective analyzers in which the control compositions have a relatively low IRF, and are prepared from a aminoglycoside compound containing loading agent delivery solution.

FIGS. 4a and 4b illustrate scattergrams to show likely expected results from a stabilized RBC material.

FIGS. 5a-5c are illustrative scattergrams to show likely expected results from different respective analyzers in which the control compositions have an intermediate level of IRF.

FIG. 6 is an illustrative scattergram showing an IRF scatter on one particular analyzer.

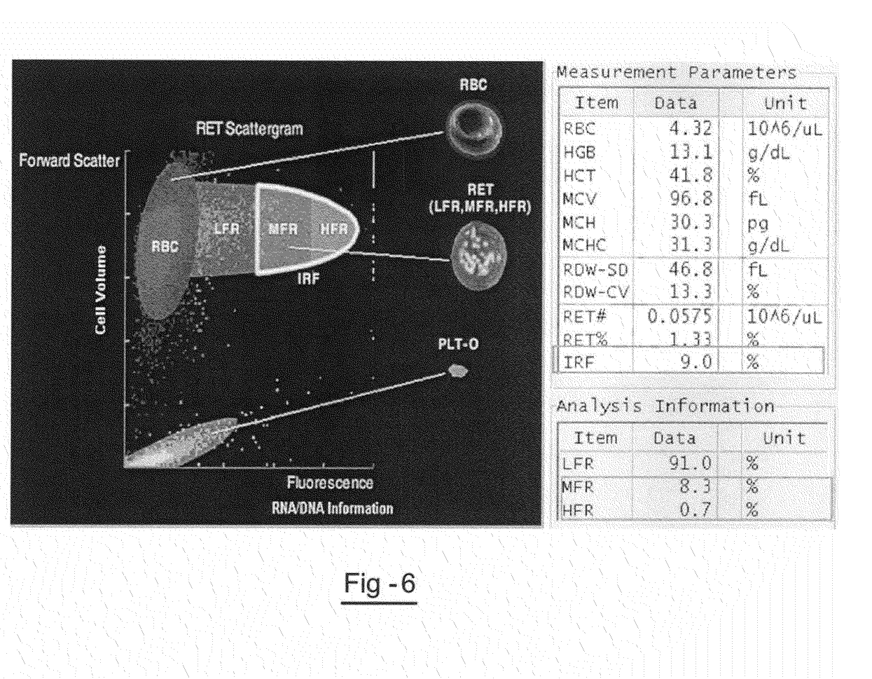

Though the scattergrams in the drawings also correspond generally and respectively with Examples 1-5 (hereafter), they are believed generally illustrative and consistent with analytical results obtainable by varying one or more of the processing parameters of those examples.

### DETAILED DESCRIPTION

In more detail, the present teachings pertain to compositions and associated methods that are adapted to provide a consistent and reproducible control system that simulates one or more detectable characteristics of a reticulocyte population, and particularly an IRF. The system contemplates a relatively long-term storage stable control composition (e.g., consistent and reproducible results are achievable for at least 12 hours, 24 hours, 48 hours, 72 hours, 1 week, 30, days, 45 days, 60 days, 90 days or longer from the time of manufacture) that employs stabilized blood cells defining simulated reticulocytes that include ah outer membrane layer that substantially encapsulates an amount of a loading agent (e.g., RNA or any other polyanionic compound capable of binding the instrument stain) selected for simulating the size stainability and morphology of reticulocytes in an IRF.

In a first aspect, the teachings herein pertain to a control composition comprising a plurality of treated red blood cells for simulating an immature reticulocyte fraction of whole blood when processed as a sample in an automated analyzer capable of detecting reticulocytes. The treated red blood cells that are employed to make the simulated reticulocyte cells herein may be of human red blood cell origin. It is possible that non-human blood cells may be employed as a starting material also. For example, one or more of bovine, porcine, or other suitable animal red blood cells may be employed as starting materials.

As will be discussed herein, it is one object of the teachings to prepare a synthetic and storage stable cell (also known as an analog) to simulate a reticulocyte in relevant detectable characteristics so that the resulting prepared cell will be detected by an automated blood analyzer as a reticulocyte. The starting material typically will be a blood cell (e.g., one that includes a certain amount of hemoglobin). The blood cell itself may generally be essentially free of any detectable amounts of ribonucleic acid (RNA). However, it should be borne in mind that individual red blood cells processed under the present teachings will typically be derived from, a supply of red blood cells (e.g. a supply of human red blood cells). Within such supply, there may incidental amounts (e.g., less than about 1% by number) of reticulocytes from the supply.

The individual treated red blood cells in the starting material thus generally will not resemble reticulocytes, let alone IRF, which (depending, upon its maturity level) may have a wide range of RNA content, across a population of such cells. That is, when starting materials are passed through an automated analyzer, they are not detected by the analyzer as a reticulocyte, let alone a reticulocyte that is from an IRF. Accordingly, one of the difficulties faced is to simulate an IRF, by manipulating the cell structure of the starting material to be of suitable size and to either encapsulate or have a membrane structure that is capable of accepting a detection agent, such as a stain, dye or other agent, in a manner that resembles how an IRF would accept such detection agent.

One approach to achieving such a structure is to manipulate a cell structure, such as a red blood, cell structure, and to enclose a loading agent within the membrane of the cell. One surprising aspect of the teachings herein is that a relatively large amount of a loading agent can be encapsulated within a membrane, of a cellular starting material. Another surprising aspect of the teachings herein is that, even though a small amount of hemoglobin may be lost during processing, the steps herein generally will contribute toward minimizing any such loss. Accordingly, final cells processed in accordance with the present teachings will typically include a cell membrane from a starting bipod cell material, an amount of a loading agent for simulating an amount of RNA expected within an immature reticulocyte, and an amount of hemoglobin (e.g., an amount of hemoglobin from the starting material blood cell). The amount of hemoglobin thus may be naturally occurring (e.g., human red blood cell hemoglobin, when the starting materials are human red blood cells). Though the cells may lose certain amounts of hemoglobin, the cells when employed in a resulting control composition generally will be sealed. Thus, during the period of control composition stability, the resulting control composition may desirably remain substantially free of free hemoglobin loss.

For purposes herein, the term “loading agent” includes one of more agents that may be naturally occurring, synthesized or a combination thereof, and which is capable of being introduced across a cell membrane into an interior volume of a cell, and which thereafter effectively resembles a content of RNA within the cell that would be encountered with naturally occurring reticulocytes. Loading agents may include a bio-polymer, an oligomer, or some other macromolecular structure. Loading agents may include two or more repeating units, which may include an electrolyte group. Examples of loading agents, may include RNA or any other polyanionic compound capable of binding the instrument stain or any combination thereof. In accordance with the teachings herein, one or more loading agent typically will be present within an interior volume of a red blood cell. RNA may come from any suitable source. By way of example RNA may come from yeast, such as Torula yeast. It may come from a plant, from bacteria, from a human tissue and/or cell source, an animal source or otherwise.

As indicated, control compositions of the teachings herein (e.g., compositions that include a population of processed cells for simulating immature reticulocytes, and which optionally may include other simulated blood cell components such as simulated mature reticulocytes) have generally long term stability. Such stability may be in a refrigerated condition, or in the absence of refrigeration (e.g., at about room temperature). For example, samples obtained from such storage stable composition, when analyzed using the same instrument may exhibit substantially consistent values (e.g., with a variation of less than about ±20%, 15% or even 10%) after the designated time, as compared with the values obtained at the time of manufacture.

Control compositions of the present teachings may include a simulated IRF component. They may include one or more additional components for simulating one or more other blood cell components (e.g., a simulated mature reticulocyte component, a white bipod cell simulated component, a simulated platelet component, a simulated nucleated red blood cell component, or otherwise). In addition to the simulated cellular components, a control composition in accordance with the present teachings may also include one PC more diluents. The diluents, as will be discussed, may include at least one stabilizing agent (in a sufficient amount for achieving the desired stability).

The stabilizing agent may be selected from a suitable carboxylic acid. For example, it may be selected, from a salicylic acid (e.g., sulfasalazine (such as in an amount of about 1 to about 25 mg %, e.g., about 10 mg %), 5-amino salicylic acid or a combination thereof). It may be a formaldehyde donor such as diazolidinyl urea, it may be selected from one or more of [[[(2-dihydro-5-methyl-3(2H)-oxazolyl)-1-methylethoxy]methoxy]methoxy]methanol (e.g., NuoSept 145), sodium hydroxymethylglycinate (e.g., Suttocide or Nuosept 44), an agent including one or more derivatives of or ingredients having 4,4-Dimethyl-1,3-oxazolidine (e.g. Oxaban A, Nuosept 101 or Nuosept 166) or any combination thereof. Examples of particular preferred agents include [[[(2-dihydro-5-methyl-3(2H)-oxazolyl)-1-methylethoxy]methoxy]methoxy]methanol (e.g., NuoSept 145), which may be employed in an amount of about 2 to about 8 ml/l of the post-encapsulation solution. Another is sulfasalazine, which may be employed in an amount of about 0.01 to about 0.25 g/l, e.g., about 0.10 g/l of the post-encapsulation solution. Any combination of the above stabilizing agents may be employed.

The amount of the simulated cellular components will be some predetermined amount that can be used as a reference value. For instance, the reference value may be one or more amounts that represent a known amount of reticulocytes that would correspond with a normal amount of reticulocytes in an IRF, a high amount of reticulocytes in an IRF, a low amount of reticulocytes in an IRF, an intermediate amount of reticulocytes in an IRF, or some other value. In one illustrative composition, simulated components may be present in an amount for resembling a reticulocyte population having an amount of reticulocytes corresponding with an IRF in a relatively low range. By way of example, for some analyzers, it may be an amount that is reported as about 0.05 to about 0.3 (i.e., about 5 to about 30%), of about 10 to about 30%, or about 35 to about 65% of the total amount of cells detected by an analyzer as reticulocytes. In one illustrative composition, simulated components may be present in an amount for resembling a reticulocyte population having an amount of reticulocytes corresponding with an IRF in a relatively high range. By way of example, for some analyzers, it may be an amount that is reported as about 0.30 to about 0.65 (i.e., about 30 to about 65%)) of the total amount of cells detected by an analyzer as reticulocytes. A composition may have an intermediate amount of reticulocytes, such as one having a reported value between the above ranges. The amount of IRF reported may be instrument specific, so the above ranges are not necessarily universal in their application to the present teachings.

What is contemplated, however, is that the teachings herein envision embodiments in which first and second simulated IRF components ere prepared and employed, respectively, in at least two compositions, each yielding a generally consistent and reproducible reporting of different relative (e.g., low, high, and optionally intermediate) IRF values.

Turning now to more specific details about the manner in which the simulated reticulocytes and compositions containing them herein are made, in general they include, steps of preparing a supply of red blood cells for processing, removing hemoglobin from red blood cells from the supply, rendering the membranes of the red blood cells permeable, transporting an amount of a loading agent across the membranes (e.g., via pores formed in the membranes), sealing the membranes after the loading agent is within an interior volume of the cells, and optionally stabilizing the cells (e.g., using the fore-mentioned stabilizing agent).

Initial Cell Stress:

A supply of blood cells is provided. For example, a supply of human blood is provided, such as in a form of one or more red blood cell packs. Red blood cells may be treated in one or more initial cell stress steps. In any such steps the cells (e.g., red blood cells from the supply of cells) are treated in a manner so that younger cells or more particularly cells with relatively stronger membrane structures are separated, from older and/or weaker cells. In this manner, in subsequent processing the red blood cells are largely tolerant, to the osmotic, variations that will result. One approach to this is to store a population of red blood cells in a diluent formulated so that older cells, weaker cells or both are selectively lysed. The diluent may be a suitable stress solution (e.g., a hypotonic stress solution) that is capable of selective destruction of weak or aged red blood cells within a sample, while leaving more viable and robust cells in tact. The diluent may be employed in any suitable amount and for a time sufficient for selectively destroying weakened or aged red blood cells within the supply. One example of such a stress solution may include one, two, three or more biocidal agents. The biocidal agents may be employed in an amount of at least about 1 g/l, 5 g/l or even 10 g/l of the solution. The biocidal agents may be employed in an amount of less than about 50 g/l, 35 g/l or even 25 g/l of the solution. The solution may include one or more agents for affecting osmotic strain on a cell membrane. For example it may include one or more polyethers (e.g., polyethylene glycol (“PEG”), such as PEG having a molecular weight of about 20,000), and one or more salts (e.g., NaCl). Any such polyether may be present in an amount of at least about 8 g/l, 11 g/l or even 15 g/l of the solution. The polyether may be present in an amount of less than about 50 g/l, 35 g/l or even 25 g/l of the solution. An example of a suitable stress solution is in the following Table 1.

TABLE 1

| | | |
|---|---|---|
| Polyethylene glycol (MW 20,000) | 15.0 | g/l |
| NaCl | 5 | g/l |
| Methyl paraben | 0.4 | g/l |
| Chloramphenicol | 0.15 | g/l |
| Neomycin | 0.4 | g/l |

A volume of about 1 part by volume of cells to about 2 parts by volume of the stress solution may be employed.

After a sufficient period of time in the diluent for achieving suitable lysis (e.g., about 24 to about 48 hours), the remaining viable cells are separated from the lysed cells and any remaining leukocytes, by a suitable separation process. For example, they may be passed through one or more leukocyte filters, under suitable aseptic conditions at about room temperature (e.g., about 20 to about 24° C.). The remaining viable cells, after the separation, are then concentrated. They may be centrifugated, such as by subjecting them to centrifugation at about 500 to about 750×g (e.g., about 657×g) for a suitable period of time, such as for about 5 to about 25 minutes (e.g., about 15 minutes).

Pre-Permeabilization:

After any initial cell stress step, the cells are diluted to a red blood cell count of about 1×10⁶/μl to about 3×10⁶/μl, e.g., about 2×10⁶/μl, in a suitable preservative diluent having a pH of about 7.1 and an osmolality of about 300 to about 320 mOsm/kg. They are stored in the diluent for a period of about 5 to about 20 days. An example of one such diluent includes the ingredients of Table 2.

TABLE 2

| | | |
|---|---|---|
| EPTA(disodium salt) | 7.04 | g/l |
| Magnesium gluconate | 3.92 | g/l |
| Sodium phosphate (dibasic) | 2.68 | g/l |
| Polyethylene glycol | 7 | g/l |
| (MWₐᵥ = 20,000) | | |
| Methyl paraben | 0.4 | g/l |
| Neomycin sulfate | 0.4 | g/l |
| Chloramphenicol | 0.15 | g/l |
| Glucose | 6 | g/l |
| inosine | 1 | g/l |

Approximately 12 to about 36 hours (e.g., about 24 hours) prior to introducing the loading agent into to cells, they are concentrated again, such as by subjecting them to centrifugation at about 500 to about 750×g (e.g., about 657×g) for a suitable period of time, such as for about 5 to about 25 minutes (e.g., about 15 minutes). They are also washed one or more times. For example, they are washed two times, three times or more with equal volumes of a suitable generally isotonic solution (which may include, one or more, antimicrobials) having a pH of about 7.2 to about 7.5 and more preferably about 7.3 to about 7.4. The solution preferably has an osmolality of about 270 to about 310 mOsm/kg, and more preferably about 280 to about 300 mOsm/kg. For example, they are washed with a generally isotonic sodium chloride solution having the ingredients and approximate concentration of Table 3:

TABLE 3

| | | |
|---|---|---|
| Sodium chloride | 8.7 | g/l |
| Neomycin sulfate | 0.4 | g/l |
| Methyl paraben | 0.4 | g/l |
| Chloramphenicol | 0.15 | g/l |

After washing, the cells are packed to a hematocrit of about 60 to about 90%, and more preferably about 70 to about 80% where they remain (e.g., for an overnight period) until the steps of introducing loading agent therein.

Permeabilizing Cells:

Among the unique features of the present invention is that the use of certain reagents allows for a consistent and reproducible ability to manage pore size formation in a membrane of a red blood cell so that a loading agent can be introduced (without damage to the membranes) within an interior volume of the blood cell in sufficient amount for simulating the amounts of RNA that naturally occur in typical reticulocytes of an IRF. Accordingly, one approach herein contemplates the use of a generally hypertonic solution that contains DMSO and includes an amount (e.g., less than about 50 vol %) of a slightly hypotonic solution, and particularly a HEPES buffered solution. The HEPES buffered solution may have a pH ranging from about 7.3 to about 7.6, and more preferably about 7.4 to about 7.5. It may have an osmolality of about 260 to about 300 mOsm/kg, and more preferably about 270 to about 290 mOsm/kg. The HEPES buffered solution may include HEPES and may also include one or more electrolytes, one or more antimicrobials, or both. An example of a suitable HEPES buffered solution is in Table 4.

TABLE 4a

| | | |
|---|---|---|
| KCl | 9.95 | g/l |
| HEPES | 2.38 | g/l |
| NaCl | 0.58 | g/l |
| MgCl₂ | 0.19 | g/l |
| CaCl₂ | 0.00111 | g/l |
| Methyl paraben | 0.4 | g/l |
| chloramphenicol | 0.15 | g/l |

The solution of Table 4 or other suitable solution may be combined with one or more other ingredients for forming a hypertonic solution that is employed herein as a permeabilizing solution (e.g., a solution having an osmolality of greater than about 700 mOsm/kg, or even greater than about 850 mOsm/kg). The solution may have, ah osmolality of greater than about 1000 mOsm/kg, more preferably greater than about 2500 mOsm/kg, still more preferably greater than about 5000 mOsm/kg, and even possibly greater than about 7500 mOsm/kg. For example, one preferred hypertonic solution will have an osmolality of about 8700 to about 9100 mOsm/kg. The hypertonic solution may be slightly basic. For example, it may have a pH of about 7.6 to about 8 (e.g., about 7.75 to about 7.85). The hypertonic solution may include a suitable amount of a suitable aprotic solvent. The hypertonic solution may include an organo-sulfur compound. An example of a suitable ingredient for the hypertonic solution is dimethyl sulfoxide (DMSO). The permeabilizing solution may thus include at least about 50 vol %, at least about 60 vol % (e.g., about 60.16%) or more of DMSO. The permeabilizing solution may include the DMSO in combination with the solution of Table 4a.

For example, the permeabilizing solution may include DMSO and a buffered solution so that it has the composition of the following Table 4b:

TABLE 4b

| | |
|---|---|
| Hepes buffered solution of Table 4a | 451.4 g/l |
| Dimethyl sulfoxide (DMSO) | 601.6 g/l |

One or more loading agent delivery solutions desirably are employed in an amount and of a type sufficient for causing a rapid transport of loading agent through open pores in a cell membrane (e.g., pores opened during permeabilizing) and a rapid subsequent re-sealing of the cell membrane to close the pores after the rapid transport has occurred. The loading agent delivery solution will typically be a generally hypotonic solution that is capable of avoiding any deleterious reaction with the loading agent, the cell membrane of the treated red blood cells into which the loading agent is introduced, or more preferably both. The loading agent delivery solution also is such that it can be used in sufficient amounts that, following its introduction into a solution containing cells having been permeabilized with a hypertonic solution, the loading agent delivery solution will counteract the permeabilization reaction, effectively arresting it. It will also cause restoration of the membrane structure of the cells substantially to a sealed state.

The loading agent delivery solution may be a generally aqueous solution that includes a loading agent. It may include one or more antimicrobials along with an amount of loading agent.

Optionally, it may include one or more amine-containing compounds. For example, (and particularly as may be employed for controlling the extent of a simulated IRF formation in a population of simulated reticulocytes), it may include at least one of an aminoglycoside (e.g., neomycin sulfate), a tertiary amine such as triethanolamine, a primary amine such as 2-amino-2-hydroxymethyl-propane-1,3-diol (tris), N-tris[hydroxyl methyl]methyl-3-aminopropanesulfonic acid (TAPS), any salt or other derivative of any of the above, or any combination thereof.

The loading agent delivery solution may have a pH of about 7.4 to about 7.8 (e.g., about 7.5 to about 7.7). It may have an osmolality of about 170 to about 250 mOsm/kg (e.g., about 190 to about 230 mOsm/kg). Two or more different loading agent delivery solutions may be used, such as one producing a composition to simulate about 15 to about 30% (e.g., relatively low) IRF, and another to simulate about 50 to about 65% (relatively high) IRF.

By way of example, one possible loading agent delivery solution includes a loading agent such as RNA (e.g., RNA derived from a non-human source, such as RNA from Torula yeast), present in a solution in a mass concentration in amount of about 5 to about 100 g/l, and more preferably about 10 g/l to about 80 g/l (e.g., about 50 g/l) in an aqueous solution that includes a tris-containing compound in an amount of from 1 to about 50 g/l of solution. By way of illustration, it may be employed in an amount of about 10 to about 50 g/l (e.g., about 24 g/l tris), and about 0.2 to about 1 g/l tris-HCl (e.g., about 0.54 g/l).

One of the surprising aspects of the present teachings is that the loading agent delivery solution that is selected can be employed to provide simulated reticulocyte cells that exhibit consistent and reproducible, quantities of a reticulocyte population having a known range of cells for simulating an IRF. Yet another surprising aspect is that a selection as between certain ingredients in the loading agent delivery solution can yield consistent and reproducible quantities of a reticulocyte population having a first known range of relatively low amounts of cells for simulating an IRF or a second known range of relatively high amounts of cells for simulating an IRF. Without intending to be bound by theory, it is believed that certain ingredients unexpectedly, but consistently and reproducibly, affect the ability of the loading agent, once encapsulated according to the present teachings, to bind with a dye to which the loading agent is exposed during operation, of an automated analyzer. As a result of the surprising features of this, aspect, it is possible to prepare (with precision previously unobtainable) multiple, batches of simulated reticulocytes, with each batch having relatively known IRF amount, but which can be controlled to differ consistently and reproducibly with the manufacture of the batches.

In general, this aspect of the teachings may be predicated upon using a loading agent delivery solution that includes at least one tris compound, for preparing a reticulocyte population that has an IRF below about 30%. For example, tris compounds in an amount greater than about 30 g/l, or even 50 g/l may be employed for preparing a reticulocyte population that has a relatively low IRF. For example to prepare a one liter aqueous solution having about 50 grams (g) of yeast RNA, an amount of at least about 20, 24 or even 28 g of tris compounds may be employed.

This aspect of the teachings may be predicated upon using a loading agent delivery solution that includes an aminoglycoside (e.g., neomycin sulfate or (1R,2R,3S,4R6S)-4,6-diamino-2-{[3-O-(2,6-diamino-2,6-dideoxy-β-L-idopyranosyl)-β-D-ribofuranosyl]oxy}-hydroxycyclohexyl 2,6-diamino-2,6-dideoxy-α-D-glucopyranoside), in an amount greater than about 1 g/l, 3 g/l or even 5 g/l, for preparing a reticulocyte population that has an IRF below about 30%. For example to prepare a one liter aqueous solution having about 50 grams (g) of yeast RNA, an amount of at least about 1 g, 3 g or even 5 g of the aminoglycoside may be used.

This aspect of the teachings may be predicated upon using a loading agent delivery solution that is essentially/free of any aminoglycoside (e.g., it has less than, about 0.7 g/l), and/or is essentially free of any tris-containing compound (e.g., it has less than 10 g/l of any tris-containing compound) for preparing a reticulocyte population that has a relatively high IRF.

The following Table 5a is an example of a loading agent delivery solution for producing a batch of loading agent encapsulated cells to simulate relatively low IRF. The following Table 5b is ah example of another loading agent delivery solution for producing a batch of loading agent encapsulated cells to simulate relatively low IRF. The following Table 5c is ah example of a loading agent delivery solution for producing a batch of loading agent encapsulated cells to simulate relatively high IRF.

TABLE 5a

| | | |
|---|---|---|
| Water | 1 | liter (l) |
| Trizma Base | 24 | g/l |
| Tris-HCl | 5.4 | g/l |
| Torula yeast RNA | 50 | g/l |
| Neomycin sulfate | 0.4 | g/l |
| Methyl paraben | 0.4 | g/l |
| Chloramphenicol | 0.15 | g/l |

TABLE 5b

| | | |
|---|---|---|
| Water | 1 | L |
| 10N sodium hydroxide | 12.5 | ml/l |
| Torula yeast RNA | 50 | g/l |
| Neomycin sulfate | 5.4 | g/l |
| Methyl paraben | 0.4 | g/l |
| Chloramphenicol | 0.15 | g/l |

TABLE 5c

| | | |
|---|---|---|
| Water | 1 | L |
| 10N sodium hydroxide | 12.5 | ml/l |
| Torula yeast RNA | 50 | g/l |
| Neomycin sulfate | 0.4 | g/l |
| Methyl paraben | 0.4 | g/l |
| Chloramphenicol | 0.15 | g/l |

Turning further to the processing of cells to introduce the loading agent into a volume within a cell membrane, for the introduction of the loading agent into red blood cells, the packed red blood cells (e.g., those that were held overnight) are diluted with about 0.05 to about 2 (e.g., about 0.1) parts by volume of the permeabilizing solution and about 3 to about 5 (e.g., about 4) parts by volume of the loading agent delivery solution with each other. The red blood cells are suspended in the generally isotonic solution to a concentration of about 8.0×10⁶/μl or a hematocrit of 70-80%. The permeabilizing solution is first added to the suspended red blood cells. The resulting solution is allowed to stand at a suitable temperature and for a suitable time to form pores of sufficient size in the red blood cell membranes to allow entry of the loading agent into to cells, but without permanent degradation of the cell membranes (e.g., at about room temperature for about 5 to about 20 minutes, and more particularly about 10 minutes). Alternatively stated, the time, temperature and relative concentrations of the generally isotonic solution and the permeabilizing solution is sufficient so that the osmolality of the resulting solution increases to a value in the range of about 800 to about 1400 mOsm/kg (e.g., about 1100 mOsm/kg), or to some other value that allows the membranes to become permeable to the loading agent, and accordingly permits entry of the loading agent into the interior volumes of the cells.

Loading and Re-Sealing of Cells:

After sufficient time has elapsed, so that the cells are permeabilized, the loading agent delivery solution is introduced into solution that includes the generally isotonic solution and the permeabilizing solution, in the above mentioned amounts. The loading agent delivery solution is rapidly introduced, while mixing with the generally isotonic solution and the permeabilizing solution. Loading agent from the loading agent delivery solution is able to pass through a cell membrane via a pore from the permeabilizing step. The tonicity of the loading agent delivery solution also causes/the pores along the membrane of the cell to close, thereby trapping and encapsulating the loading agent within the membrane. This loading agent transport and membrane re-sealing phenomena, happens relatively rapidly, providing an added benefit that a relatively large amount of hemoglobin is retained within the cell membrane. By virtue of the rapid introduction and the resulting osmotic shock it is possible to introduce the loading agent info the interior volumes of the cells, and substantially instantaneously cause pore closing, and thus re-sealing of the membranes, so that the loading agent remains encapsulated within the cells.

The rapid membrane restoration that results from the above process helps to assure substantially inconsequential loss of hemoglobin within the red blood cells, while helping to assure the mean cellular volume (MCV) of the cells is approximately the same as the original MCV value.

Within about 5 minutes following the rapid introduction step, the suspension is incubated at about room temperature for about 60 to about 90 minutes. The red blood cells are then subjected to centrifugation at about 300 to about 500×g (e.g., about 420×g) for a suitable period of time, such as for about 5 to about 25 minutes (e.g., about 15 minutes). They are then washed. For example, they are washed in three volumes of a suitable post-encapsulation solution multiple times (e.g., three times). The post-encapsulation, solution may have a pH of about 7.2 to about 7.6 (e.g., about 7.4). It may have an osmolality of about 295 to about 335 (e.g., about 305 to about 325) mOsm/kg. It may include one or more of the stabilizing agents described herein.

The post-encapsulation solution may in include a halide salt (e.g., sodium halide salt, such, as sodium fluoride), in ah amount sufficient that upon dissociation in the solution, one or more of its ionic components will stabilize one or more components of the resulting composition. For example, salt may be employed in an amount of about 0.05 to 1 g/l (e.g., about 0.5 g/l).

An example of a suitable post-encapsulation solution is described in the following Table 6.

TABLE 6

| | | |
|---|---|---|
| Polyethylene glycol | 7 | g/l |
| Disodium EDTA | 11.73 | g/l |
| Magnesium gluconate | 6.53 | g/l |
| Sodium phosphate | 4.47 | g/l |
| Glucose | 10 | g/l |
| Inosine | 0.25 | g/l |
| Sulfasalazine | 0.1 | g/l |
| Sodium fluoride | 0.5 | g/l |
| Pluronic F-88 | 1 | g/l |
| Chloramphenicol | 0.15 | g/l |
| Neomycin sulfate | 0.4 | g/l |
| Methyl paraben | 0.4 | g/l |

During this step, supernatant containing hemolysate and excess loading agent is discarded. Cells may be then re-suspended into the post-encapsulation solution and incubated at a suitable temperature and time (e.g., about 48 to about 72 hours at a temperature of below about 10° C., such as about 6° C.) to cause remaining cells that are relatively weak to lose hemoglobin resulting in a loss in density and removal through subsequent washing. The cells are then washed to remove free hemoglobin and any of the remaining damaged cells. They are resuspended in a solution having the composition of Table 6.

Further stabilization may be performed by washing the RNA encapsulated RBCs three time into the solution identified in Table 6 that also contains 0.4% Nuosept 145. Other Nuosept compounds such as 44, 101, and 166 or diazolidinyl urea (DU) may also be used at comparable concentrations. After the third resuspension in the Nuosept of DU containing solution, the cell count is adjusted to 2.0×10⁶/μl and stored at room temperature for 3 to 4 days. After remaining in fix for the designated time, the cells, are washed three times in the solution identified in Table 6.

In other aspects, the teachings herein pertain to a reference control for assuring consistent and reproducible values for simulating an immature reticulocyte fraction of whole blood. Controls in accordance with the present teachings may be stand-alone reticulocyte controls (e.g., a control may consist essentially of simulated reticulocytes of an IRF, or a control may consist essentially of simulated mature reticulocytes in combination with simulated reticulocytes of an IRF, both being without any other simulated blood cell component). Controls in accordance with the present teachings may include other simulated components for a multi-parameter blood cell control, e.g., components for simulating a blood cell component such as a platelet, one two or more white blood cell subpopulations, erythroblasts, or any combination). Examples of multi-parameter controls or components with which the cells of the present teachings may be combined include, without limitation, those illustrated in U.S. Pat. No. 7,618,821; 6,200,500; 6,403,377; 5,731,205; 5,008,201; 5,432,089; or 6,653,137.

The teachings also pertain to use of such a control, such as in a method for determining the accuracy and reproducibility of the operation of an analytical instrument capable of measuring immature reticulocyte fraction. For example, such method may include steps of: passing a known quantity of a control through, an automated analyzer adapted for measuring immature reticulocyte fraction; determining the immature reticulocyte fraction level in said control using the instrument; and comparing the immature reticulocyte fraction level obtained with its known reference quantity to ascertain if the instrument is properly functioning.

Another contemplated use of the present teachings envisions a method for identifying a condition indicated by an abnormal presence of immature reticulocyte fraction, comprising the steps of 1) passing a sample of patient blood through an analyzer that detects reticulocytes; 2) compiling patient blood sample information about the presence of reticulocytes in the patient blood sample, using the analyzer; 3) passing at least one sample of at least one control composition according to the present teachings through the same analyzer; 4) compiling control composition sample information about the presence of immature reticulocyte fraction in the control composition; 5) comparing the patient blood sample information with the control composition sample information to identify the extent of overlap; and 6) optionally, reporting the results of the comparing step. The step of passing at least one sample of at least one control composition may include a step of passing at least one sample of a first control composition having a first predetermined quantity of simulated immature reticulocytes, and passing at least one sample of at least one second control composition having a second predetermined quantity of simulated immature reticulocytes that differs from the first predetermined quantity. The step of reporting the results may be performed by a computer. The step of reporting the results may be performed by the analyzer.

By way of example, to illustrate the methods herein, one or more control composition may be prepared in a manner so that one or more respective known amounts of simulated immature reticulocytes are present in the composition, and/or so that one or more respective known amounts of overall simulated reticulocytes are present in the composition. For instance, there may be a kit that includes a control composition with a relatively low known amount by number and a relatively high known amount by number of simulated immature reticulocytes. There may be a kit that includes a control composition with a relatively low known amount, an intermediate known amount and a relatively high known amount of simulated immature reticulocytes. There may be a kit that includes a control composition with a relatively low known amount by number (e.g., about 3 to about 5%) of overall reticulocytes (in the total reticulocyte and red blood cell populations), a relatively intermediate known amount (e.g., about 6 to about 15%) of overall reticulocytes, and a relatively high known amount (e.g., about 16 to about 30%) of overall reticulocytes. There may be a kit that includes a control composition with a relatively low known amount (e.g., about 3 to about 5%) of overall reticulocytes, and a relatively high known amount (e.g., about 16 to about 30%) of overall reticulocytes.

The kits may be fun consecutively through an analyzer in order to ascertain the nature of the readout the analyzer is providing for the known amounts of the simulated immature reticulocytes. The analyzer should report different information for each of the different known amounts. For example (with arbitrary values in the following for illustration), it might report a first overall reticulocyte value of 6% and a first IRF quantity value 0.6 for a sample with a relatively high known amount of IRF. It might report a first overall reticulocyte quantity value of 6% and a first IRF quantity value of 0.25 for a sample with a relatively low known amount.

Within a certain predetermined time (e.g., within about 1 week, within about 72 hours, within about 48 hours, within about 24 hours, within about 12 hours, within about 6 hours or even within about 1 hour) of running the control compositions of the kits through the analyzer, a patient sample may be run through the analyzer. Suppose the patient sample has an overall reticulocyte amount value of about 6% and an amount of IRF of about 0.6, the step of comparing the sample information with information about the control composition might result in the identification of a similarity as between the sample and the high known value control composition, or a report that identifies the proximity of the value obtained relative to the known value. The comparison step could be performed with suitable software. It is contemplated that the software would perform the comparison and assign a range of values to the control composition. For example, if a high known amount was 0.6, then it might compare the patient sample, and if the patient sample is within a certain amount above or below the 0.6 value (e.g., above a value of 0.45, or some other value that may be established by a user), then it could issue a warning and report the value obtained and the fact that the value is in a range associated with a high known value. Thus, for the above example, a patient value of 0.55 might be reported along with the flag that warns such value to correspond with a relatively high IRF.

### EXAMPLES

The following examples illustrate aspects of the teachings herein. Comparable results are expected when substituting other alternative ingredients disclosed in the present teachings. Furthers comparable results are believed possible when employing amounts within about ±10% of the stated values. Further comparable results are believed possible when employing an alternative loading agent other than the RNA, or in addition to the RNA of the following examples.

As will be seen from the Examples that follow, a similarly prepared composition, when run on different instruments is expected to generate different IRF values. However, as also seen, as among the different instruments, a similarly prepared composition having a relatively low known range of IRF consistently and reproducibly is reported as having a lower IRF value than a similarly prepared composition having a relatively high known range of IRF. Further, though not illustrated in the following, similar results that consistently and reproducibly achieve comparatively higher or lower IRF values are expected for the Coulter LH-series instruments.

### Example 1

This example describes the preparation of a reticulocyte component with a relatively high IRF. Human red blood cells (“RBCs”) are prepared by suspending cells in hypotonic stress solution of sodium chloride for up to 24 hours after which time the RBCs are centrifuged and the supernatant is removed. The RBCs are then suspended in a preservative solution at a count of about 2.0×10⁶/μl and filtered through a leukocyte removal filter.

The erythrocytes are diluted after filtration. In this step, filtered RBCs are concentrated by centrifugation for 15 minutes at 657×g. The RBC pellet is diluted to a RBC count of 2×10⁶/μl with a preservative diluent. The RBCs are stored in this diluent for 5-20 days prior to encapsulation.

A day before encapsulation the RBCs are concentrated by centrifugation for 15 minutes at 657×g, and washed 3 times with equal volumes of an isotonic sodium chloride solution. After washing, the cells are packed to a hematocrit of 70-80% and used for encapsulation.

After the prepared erythrocytes are stored at room temperature overnight, the encapsulation process is initiated. A volume of a DMSO-containing permeabilizing solutions (as in Table 4b) equal to 10% of the RBC volume is added to the packed RBCs (Hct=70-80%).

After about 10 minutes, about 5% by w/v of an RNA-containing loading agent delivery solution (as described Table 5c) in equal to about 4 times the original RBC volume is rapidly added to the RBC/DMSO-containing permeabilizing solution preparation.

After the encapsulation step, the suspension is incubated at room temperature for about 60-90 minutes. Next, the treated RBCs are concentrated by centrifugation for 15 minutes at 420×g, and washed with 3 volumes of final solution three times. During, this step the supernatant containing excess, hemolysate and RNA is discarded. Cells are resuspended in final solution (as in Table 6) and incubated for 48-72 hours at about 6° C. Over these 2-3 days, the weaker RBCs lose hemoglobin. The cells are subsequently washed a minimum of 3 times to remove the resulting free hemoglobin and damaged cells and resuspended in a solution as in Table 6. The cells are adjusted to the desired RBC count using the solution identified in Table 6.

The RNA encapsulated RBC material is washed three times into the solution identified in Table 6 that also contains 0.4% Nuosept 145. Other Nuosept compounds such as 44, 101, and 166 or diazolidinyl urea (DU) may also be used at comparable concentrations. After the third resuspension in Nuosept or DU containing solution, the cell count is adjusted to 2.0×10⁶/μl and stored at room temperature for 3 to 4 days. The cells are then washed three times in the solution identified in Table 6.

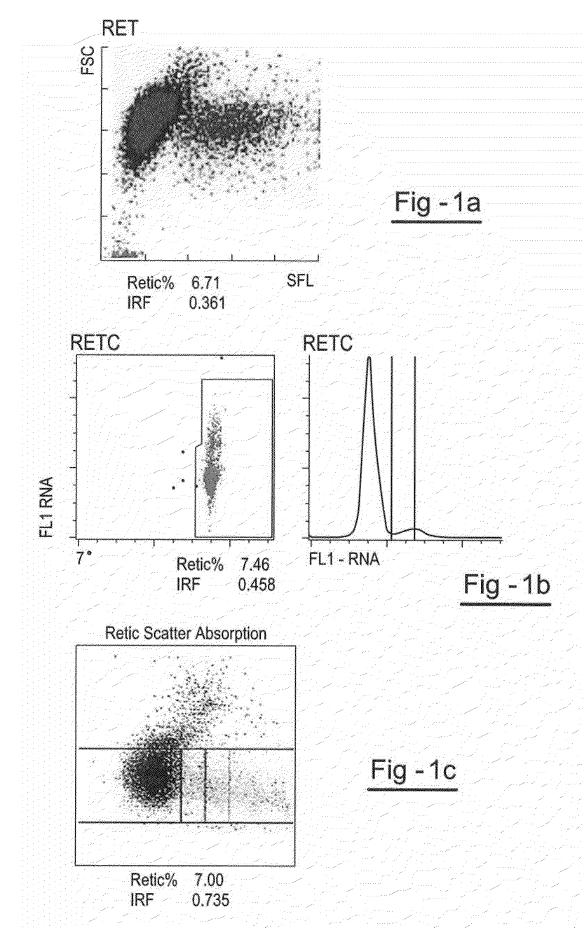

The resulting cells are expected to provide a scattergram reading on a XE-5000 instrument consistently over a period of at least about 30 days, 60 days or even 90 days, resembling that of FIG. 1a.

The resulting cells are expected to provide a scattergram reading on a Sapphire instrument consistently over a period of at least about 30 days, 60 days or even 90 days, resembling that of FIG. 1b.

The resulting cells are expected to provide a scattergram reading on a Advia 2120 instrument consistently over a period of at least about 30 days, 60 days or even 90 days, resembling that of FIG. 1c.

### Example 2

The following example describes the preparation of a reticulocyte component with a relatively low IRF. Human RBCs are prepared by suspending cells in hypotonic stress solution of sodium chloride for up to 24 hours after which time the RBCs are centrifuged and the supernatant is removed. The RBCs are then suspended in a preservative solution at a count of 2.0×10⁶/μl and filtered through a leukocyte removal filter.

The erythrocytes are diluted after filtration. In this step, filtered RBCs are concentrated by centrifugation for 15 minutes at 657×g. The RBC pellet is diluted to a RBC count of 2×10⁶/μl with a preservative diluent. The RBCs are stored in this diluent for 5-20 days prior to encapsulation.

A day before encapsulation the RBCs are concentrated by centrifugation for 15 minutes at 657×g, and washed 3 times with equal volumes of ah isotonic sodium chloride solution. After washing, the cells are packed to a hematocrit of 70-80% and used for encapsulation.

After the prepared erythrocytes are stored at room temperature overnight, the encapsulation process is initiated. A volume of DMSO-containing permeabilizing solution equal to about 10% of the RBC volume is added to the packed RBCs (Hct=70-80%).

After about 10 minutes, approximately 5% by w/v RNA in a Tris buffer solution (as described Table 5a) equal to about 4 times the original RBC volume is rapidly added to the RBC/DMSO-containing permeabilizing solution preparation.

After the encapsulation step, the suspension is incubated at room temperature for about 60-90 minutes. Next, RBCs are concentrated by centrifugation for 15 minutes at 420×g, and washed with 3 volumes of final solution three times.

During this step the supernatant containing excess hemolysate and RNA is discarded. Cells are resuspended in final solution and incubated for 48-72 hours at 6 degrees. Over that 2-3 day period, the weaker RBCs lose hemoglobin. The cells are subsequently washed a minimum of 3 times to remove the resulting free hemoglobin and damaged cells and resuspended in a solution as in Table 6. The cells are adjusted to the desired RBC count using the solution identified in Table 6.

The RNA encapsulated RBC material is washed three times into the solution identified in Table 6 that also contains 0.4% Nuosept 145. Other Nuosept compounds such as 44, 101, and 166 or diazolidinyl urea (DU) may also be used at comparable concentrations. After the third resuspension in Nuosept or DU containing solution, the cell count is adjusted to 2.0×10⁶/μl and stored at room temperature for 3 to 4 days. The cells are then washed three times in the solution identified in Table 6.

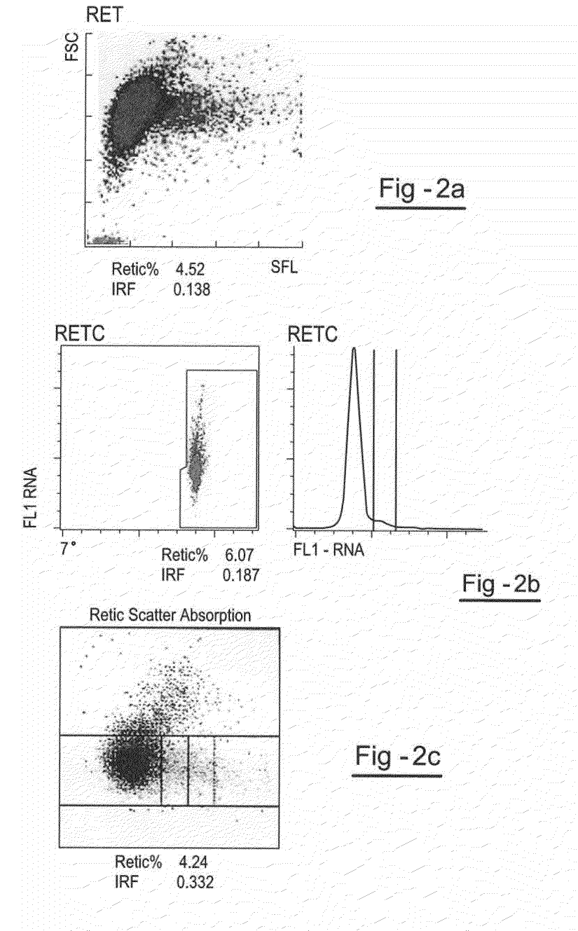

The resulting cells are expected to provide a scattergram reading on a XE-5000 instrument consistently over a period of at least about 30 days, 60 days or even 90 days, resembling that of FIG. 2a.

The resulting cells are expected to provide a scattergram reading on a Sapphire instrument consistently over a period of at least about 30 days, 60 days or even 90 days, resembling that of FIG. 2b.

The resulting cells are expected to provide a scattergram reading on a Advia 2120 instrument consistently over a period of at least about 30 days, 60 days or even 90 days, resembling that of FIG. 2c.

### Example 3

The following example describes the preparation of a reticulocyte component with a relatively low IRF. Human RBCs are prepared by suspending cells in hypotonic stress solution of sodium chloride for up to 24 hours after which time the RBCs are centrifuged and the supernatant is removed. The RBCs are then suspended in a preservative solution at a count of about 2.0×10⁶/μl and filtered through a leukocyte removal filter. The erythrocytes are diluted after filtration, in this step, filtered RBCs are concentrated by centrifugation for 15 minutes at 657×g. The RBC pellet is diluted to a RBC count of 2×10⁶/μl with a preservative diluent, the RBCs are stored in this diluent for 5-20 days prior to encapsulation.

A day before encapsulation the RBCs are concentrated by centrifugation for 15 minutes at 657×g, and washed 3 times with equal volumes of an isotonic sodium chloride solution.

After washing, the cells are packed to a hematocrit of 70-80% and used for encapsulation.

After the prepared erythrocytes are stored at room temperature overnight, the encapsulation process is initiated. A volume of DMSO-containing permeabilizing solution equal to 10% of the RBC volume is added to the packed RBCs (Hct=70-80%).

After 10 minutes, 5% RNA solution containing 0.54% by w/v Neomycin Sulfate (as described Table 5b) equal to 4 times the original RBC volume is rapidly added to the RBC/DMSO-containing permeabilizing solution preparation.

After the encapsulation step, the suspension is incubated at room temperature for about 60-90 minutes. Next, RBCs are concentrated by centrifugation for 15 minutes at 420×g, and washed with 3 volumes of final solution three times.

During this step the supernatant containing excess hemolysate and RNA is discarded. Cells are resuspended in final solution and incubated for 48-72 hours at 6 degrees. Over the 2-3 days, the weaker RBCs lose hemoglobin. The cells are subsequently washed a minimum of 3 times to remove the resulting free hemoglobin and damaged cells and resuspended in a solution as in Table 6. The cells are adjusted to the desired RBC count using the solution identified in Table 6.

The RNA encapsulated RBC material is washed three times into the solution identified in Table 6 that also contains 0.4% Nuosept 145. Other Nuosept compounds such as 44, 101, and 166 or diazolidinyl urea (DU) may also be used at comparable concentrations. After the third resuspension in Nuosept or DU containing solution, the cell count is adjusted to 2.0×10⁶/μl and stored at room temperature for 3 to 4 days. The cells are then washed three times in the solution identified in Table 6.

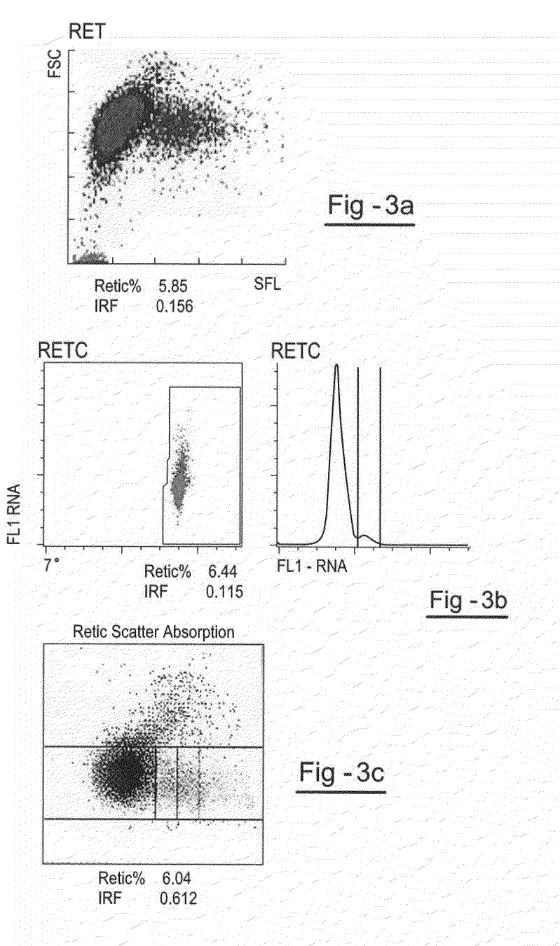

The resulting cells are expected to provide a scattergram reading on a XE-5000 instrument consistently over a period of at (east about 30 days, 60 days or even 90 days, resembling that of FIG. 3a.

The resulting cells are expected to provide a scattergram reading on a Sapphire instrument consistently over a period of at least about 30 days, 60 days or even 90 days, resembling that of FIG. 3b.

The resulting cells are expected to provide a scattergram reading on a Advia 2120 instrument consistently over a period of at least about 30 days, 60 days or even 90 days, resembling that of FIG. 3c.

### Example 4

The following example describes the preparation of normal, stabilized, non-encapsulated RBCs, such as those that may be employed in combination with the simulated reticulocytes herein, for resembling red blood dells of a sample. Human RBCs are prepared by suspending cells in hypotonic stress solution of sodium chloride for up to 24 hours after which time the RBCs are centrifuged and the supernatant is removed. The RBCs are then suspended in a preservative solution at a count of 2.0×10⁶/μl and filtered through a leukocyte removal filter. The cells can be stored for up to 30 days prior to being washed a minimum of 3 times into a solution like the post-encapsulation solution in Table 6. The cells are adjusted to the desired RBC count using the solution identified in Table 6.

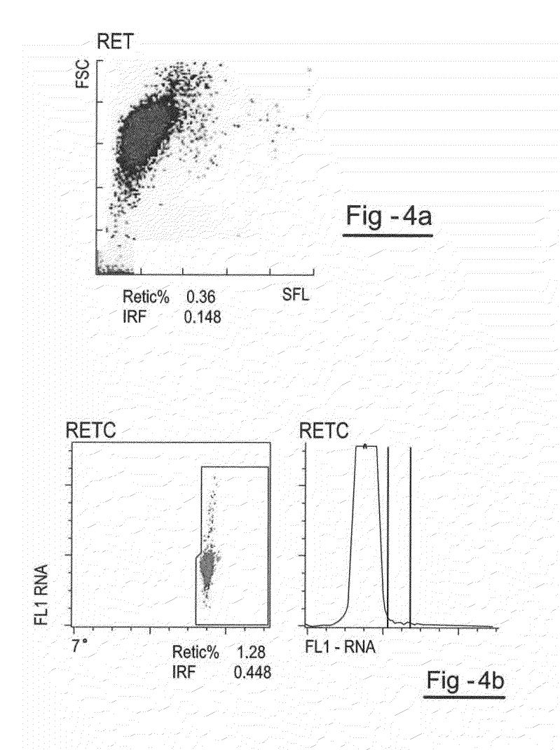

The resulting cells, are expected to provide a scattergram reading on a XE-5000 instrument consistently over a period of at least about 30 days, 60 days or even 90 days, resembling that of FIG. 4a.

The resulting cells are expected to provide a scattergram reading on a Sapphire instrument consistently over a period of at least about 30 days, 60 days or even 90 days, resembling that of FIG. 4b.

### Example 5

The following example describes blending of low and high IRF reticulocytes to produce multiple levels of IRF as a reference material. Using the low and high IRF reticulocyte preparations described in Examples 1-3, one can determine appropriate mixtures of each to prepare materials with intermediate IRFs. Any of these reticulocyte, preparations mayor may not be added, to normal, stabilized non-encapsulated RBCs (described in Example 4) to produce multi-level reticulocyte or IRF materials. The RNA encapsulation process produces reticulocyte percentages of approximately 50-80%. Low and high IRF reticulocyte preparation can be diluted to the desired percentage with RBCs. For example, to make 100 ml of material at 3×10⁶/μl at 8% reticulocytes, one would add 10 ml of 80% low IRF reticulocytes to 90 ml of the non-encapsulated RBCs. The same preparation can be made for the high IRF reticulocytes. Once the low and high IRF materials are prepared, a mid-level IRF can be made by mixing equal volumes of each.

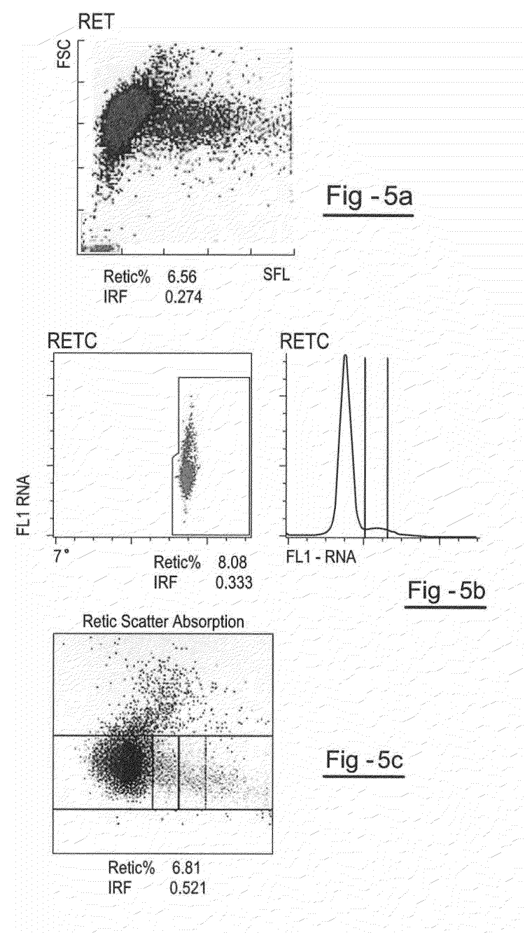

The resulting mid-level IRF samples are expected to provide a scattergram reading on a XE-5000 instrument consistently over a period of at least about 30 days, 60 days or even 90 days, resembling that of FIG. 5a.

The resulting mid-level IRF samples are expected to provide a scattergram reading on a Sapphire instrument consistently over a period of at least about 30 days, 60 days or even 90 days, resembling that of FIG. 5b.

The resulting cells are expected to provide a scattergram reading on a Advia 2120 instrument consistently over a period of at least about 30 days, 60 days or even 90 days, resembling that of FIG. 5c.

As to all of the foregoing general teachings, as used herein, unless otherwise stated, the teachings envision that any member of a genus (list) may be excluded from the genus; and/pr any member of a Markush grouping may be excluded from the grouping.

Unless otherwise stated, any numerical values recited herein include all values from the lower value to the upper value in increments of one unit provided that there is a separation of at least 2 units between any lower value and any higher value. As an example, if it is stated that the amount of a component, a property, or a value of a process variable such as, for example, temperature, pressure, time and the like is, for example, from 1 to 90, preferably from 20 to 80, more preferably from 30 to 70, it is intended that intermediate range values such as (for example, 15 to 85, 22 to 68, 43 to 51, 30 to 32 etc.) are within the teachings of this specification. Likewise, individual intermediate values are also within the present teachings. For values which are less than one, one unit is considered to be 0.0001, 0.001, 0.01 or 0.1 as appropriate. These are only examples of what is specifically intended and all possible combinations of numerical values between the lowest value and the highest value enumerated are to be considered to be expressly stated in this application in a similar manner. As can be seen, the teaching of amounts expressed as “parts by weight” herein also contemplates the same ranges expressed in terms of percent by weight. Thus, an expression in the Detailed Description of the invention of a range in terms of at “‘x’ parts by weight of the resulting polymeric blend composition” also contemplates a teaching of ranges of same recited amount of “x” in percent by weight of the resulting polymeric blend-composition.”

Unless otherwise stated, all ranges include both endpoints and all numbers between the endpoints. The use of “about” or “approximately” in connection with a range applies to both ends of the range. Thus, “about 20 to 30” is intended to cover “about 20 to about 30”, inclusive of at least the specified endpoints. Concentrations of ingredients identified in Tables herein may vary ±10%, or even 20% or more and remain within the teachings.

The disclosures of all articles and references, including patent applications and publications, are incorporated by reference for all purposes. The term “consisting essentially of” to describe a combination shall include the elements, ingredients, components or steps identified, and such other elements ingredients, components or steps that do not materially affect the basic and novel characteristics of the combination. The use of the terms “comprising” or “including” to describe combinations of elements, ingredients, components or steps herein also contemplates embodiments that consist essentially of, or even consist of the elements, ingredients, components or steps. Plural elements, ingredients, components or steps can be provided by a single integrated element, ingredient, component or step. Alternatively, a single integrated element, ingredient, component or step might be divided into, separate plural elements, ingredients, components or steps. The disclosure of “a” or “one” to describe an element, ingredient, component or step is not intended to foreclose additional elements, ingredients, components or steps. All references herein to elements or metals belonging to a certain Group refer to the Periodic Table of the Elements published and copyrighted by CRC Press, Inc., 1989. Any reference to the Group or Groups shall be to the Group or Groups as reflected in this Periodic Table of the Elements using the IUPAC system for numbering groups.

It is understood that the above description is intended to be illustrative and not restrictive. Many embodiments as well as many applications besides the examples provided will be apparent to those of skill in the art upon reading the above description. The scope of the invention should, therefore, be determined not with reference to the above description, but should instead be determined with reference to the appended claims, along with the full scope of equivalents to which such claims are entitled. The disclosures of all articles, and references, including patent applications and publications, are incorporated by reference for all purposes. The omission in the following claims of arty aspect of subject matter that is disclosed herein is not a disclaimer of such subject matter, nor should it be regarded that the inventors did not consider such subject matter to be part of the disclosed inventive subject matter.

1. A composition comprising a plurality of treated red blood. Cells for simulating a population of reticulocytes that includes an immature reticulocyte fraction of whole blood when processed as a sample in an automated analyzer capable of detecting reticulocytes.

2. A composition according to claim 1 wherein the treated red blood cells are of human red blood cells origin and include retained hemoglobin from the human blood cells.

3. A composition according to claim 1 wherein the treated red blood cells include a synthetically encapsulated loading agent.

4. A composition according to claim 1, wherein the treated red blood cells include a synthetically encapsulated polycationic loading agent capable of binding the instrument reticulocyte stain.

5. A composition according claim 1, wherein the polycation loading agent is RNA.

6. A composition according to claim 1, wherein the composition includes non-human derived RNA as the loading agent.

7. A composition according to claim 1, wherein the composition is substantially free of free hemoglobin.

8. A composition according to any one of claim 1, wherein the composition is storage stable for a period of at least 7 days.

9. A composition according to claim 1, wherein the composition includes a diluent.

10. A composition according to claim 1, wherein the composition includes a diluent that includes at least one stabilizing agent selected from [[[(2-dihydro-5-methyl-3(2H)-oxazolyl)-1-methylethoxy]methoxy]methoxy]methanol (Nuosept 145), sulfasalazine or a mixture thereof.

11. A composition according to claim 1, wherein the composition exhibits an immature reticulocyte fraction in the range of about 15-30% or about 50-65%.

12. A method for making a simulated reticulocyte comprising: contacting a suspension of a plurality of red blood cells each having a membrane in an initial state that surrounds an interior volume of a cell with ah effective amount of a hypertonic permeabilizing solution including dimethyl sulfoxide and a hypotonic loading agent delivery solution including a loading agent, for a sufficient time to form a plurality of pores in the membrane, for permitting the loading agent to enter into the interior volume of the cells, and, after entry of a desired amount of the loading agent into the interior volume of the cell, for sealing the pores for substantially restoring the membrane to the initial state while substantially encapsulating the loading agent within the resulting cell.

13. A method according to claim 12, where the method includes a step of separating a plurality of human red blood cells from a supply of human red blood cells.

14. A method according to claim 12, wherein the method includes a step of separating a plurality of human red blood cells from a supply of human red bipod cells by contacting the supply of human red blood cells with a stress solution (e.g., a solution including about 1.5 w/v % PEG (M.W. 20,000), 0.5 w/v % NaCl, 0.4% methyl paraben, 0.015 w/v % chloramphenicol, and 0.04 w/v % neomycin) in an amount and for a time sufficient for selectively destroying weakened or aged red blood cells within the supply.

15. A method according to claim 12, wherein the loading agent delivery solution is employed for providing a relatively low amount/of IRF and includes a tris compound, aminoglycoside, or both.

16. A method according to claim 12, wherein the method includes contacting the plurality of red blood cells with a substantially pH neutral and substantially isotonic preservative diluent for a period of about 5 days to about 30 days.

17. A method according to any of claim 12, wherein the method includes contacting the plurality of red blood cells with a substantially pH neutral and substantially isotonic preservative diluent for a period of about 5 days to about 30 days, the diluent including EDTA, and is held in a diluted red blood cell concentration of about 1×10⁶ to about 3×10⁶/μl.

18. A method according to claim 12, wherein the method includes packing the plurality of red blood cells to a hematocrit value of about 65 to about 85% in a unit volume of ah isotonic solution.

19. A method according to claim 12, wherein (i) the permeabilizing solution include about 0.05 to about 2 (e.g., about 0.1) parts by volume of dimethyl sulfoxide, (ii) the loading agent delivery solution is a hypotonic solution and includes about 3 to about 5 (e.g., about 4) parts by volume of a solution including a polyanionic loading agent capable of binding the instrument reticulocyte stain; or (iii) both (i) and (ii).

20. The method according to claim 12, wherein the step of contacting includes first contacting with the permeabilizing solution and then contacting with the loading agent delivery solution.
